(19) United States
(12) Patent Application Publication
(10) Pub. No.: US 2021/0253313 A1
(43) Pub. Date: Aug. 19, 2021

(54) SELF CLOSING FRAGRANCE CARTRIDGE AND FRANGRANCER

(71) Applicant: Mahle International GmbH, Stuttgart (DE)

(72) Inventors: Daniel Eberle, Ludwigsburg (DE); Sham Kasar, Pune (IN); Sven Petzner, Welzheim (DE); Oliver Schultze, Stuttgart (DE)

(21) Appl. No.: 17/163,302

(22) Filed: Jan. 29, 2021

(30) Foreign Application Priority Data
Jan. 31, 2020 (DE) 102020201192.3

(51) Int. Cl. B65D 47/20; A61L 9/12

(52) U.S. Cl. CPC B65D 47/2093; B65D 2547/06; A61L 2209/133; A61L 9/12

(57) ABSTRACT

The invention relates to a self-closing fragrance cartridge (1) for a fragrancer (20), which comprises a housing (3) having at least one housing opening (2). The housing (3) at least partially delimits a housing interior (4) on the inner side. In the housing interior (4), an actuating element (8) displaceable with respect to the housing (3) between a closed position (5) and a fragrancing position (6) and having at least one element opening (7) is at least partially arranged, which actuating element partially delimits an element interior (9). Here, in the closed position (5) of the actuating element (8) the element interior (9) is closed in a fluid-tight manner by means of the housing (3) of the fragrance cartridge (1). By comparison, in the fragrancing position (6) the housing opening (2) and the element opening (7) are arranged in a fluidically communicating manner, so that by means of the housing opening (2) and the element opening (7) the element interior (9) is connected in a fluid-permeable manner with an outer space (10) adjoining the housing (3) on the outer side.

The invention relates to a self-closing fragrance cartridge for a fragrancer and a fragrancer for dispensing a fragrance.

For the providing of a fragrance which can be delivered by means of a fragrancer, in particular to a vehicle interior, fragrance cartridges have been used for some time. In these, usually a fragrance source is present, from which the fragrance is generated. Here, conventional fragrance cartridges have a housing which delimits a housing interior receiving the fragrance source. In the housing of conventional fragrance cartridges a housing opening is provided, through which a fragrance delivered from the fragrance source can be directed out from the fragrance cartridge or respectively out from the housing interior of the housing of the fragrance cartridge. Usually, in a delivery condition, therefore before the use or respectively before the mounting of the fragrance cartridge on a fragrancer, this housing opening is closed by means of a seal or by means of a cover which is removable and thus basically at risk of being lost. To use the fragrance cartridge, such a seal must firstly be removed or penetrated in a destructive manner before mounting the fragrance cartridge on the fragrancer. A cover closing the housing opening must be removed.

It proves to be disadvantageous here that the housing opening in conventional fragrance cartridges is permanently open before the using of the fragrance cartridge. In the case where the housing opening in delivery condition of the fragrance cartridge is only closed by means of a seal, the housing opening can not be readily closed again if the fragrance cartridge has to be dismantled for maintenance purposes of the fragrancer. By comparison, in the case where the housing opening of the fragrance cartridge in a delivery condition of the fragrance cartridge is closed by means of a cover, the risk exists that this cover becomes lost, so that the housing opening of the fragrance cartridge can also then not be readily closed again. A closing of the housing opening by means of such a cover can also be simply forgotten by the user or respectively operator. This is therefore to be regarded as disadvantageous particularly because the fragrance provided by from the fragrance source of the fragrance cartridge is typically volatile and accordingly exits from the fragrance cartridge when the housing opening is not closed. In the opposite manner, through the permanently open housing opening, contaminations which are present in the environment of the fragrance cartridge can arrive into the housing interior in an undesirable manner.

It is therefore an object of the present invention to indicate new ways for fragrance cartridges, able to be used on a fragrancer, and for such fragrancers—in particular to eliminate the above-mentioned disadvantages.

This problem is solved by the subject of the independent claim 1 and the coordinate claim 15. Preferred embodiments are the subject of the dependent claims.

A basic idea of the invention accordingly is to provide a self-closing fragrance cartridge, in which an actuating element with an element opening is provided displaceably relative to a housing with a housing opening. Here, the actuating element is displaceable relative to the housing in such a way that, in a closed position, no fragrance can exit from the fragrance cartridge, and in a fragrancing position of the actuating element the housing opening with the element opening are arranged communicating fluidically in such a way that the fragrance can be delivered from the fragrance cartridge.

This offers the advantage that in the case of any dismantling of the fragrance cartridge from a fragrancer—as in the case of the maintenance of the fragrancer—the housing opening of the housing of the fragrance cartridge can be automatically closed again, so that it is ensured that for a dwell time of the fragrance cartridge in a dismantled state, no fragrance can exit unused from the fragrance cartridge and be lost. In addition, it is advantageously achieved that a manual closing of the housing opening of the fragrance cartridge can not be forgotten.

A self-closing fragrance cartridge according to the invention is suitable for use in a fragrancer. The self-closing fragrance cartridge has a housing which comprises at least one housing opening. The housing delimits a housing interior on the inner side. In the housing interior, an actuating element is at least partially arranged, which is displaceable with respect to the housing between a closed position and a fragrancing position. The actuating element at least partially delimits an element interior and has at least one element opening. In the closed position of the actuating element, the element interior is closed here in a fluid-tight manner by means of the housing of the fragrance cartridge. By comparison, in the fragrancing position of the actuating element, the housing opening and the element opening are arranged in a fluidically communicating manner. Expediently, in the fragrancing position the housing opening and the element opening are arranged in alignment with one another. A fluidically communicating arrangement of the housing opening and of the element opening in the fragrancing position can, however, also be achieved by a partial overlapping of the housing opening and of the element opening. Here, the housing opening and the element opening in the fragrancing position are arranged in a fluidically communicating manner in such a way that by means of the housing opening and of the element opening the element interior is connected in a fluid-permeable manner with an outer space adjoining the housing on the outer side. Such a fluid-permeable connection is particularly advantageously fragrance-permeable. The proposed closure mechanism of the self-closing fragrance cartridge according to the invention is advantageously able to be implemented in a structurally particularly simple manner, able to be produced at comparatively low costs and, at the same time, is particularly effective, so that it can be ensured in a particularly reliable manner that the fragrance cartridge in a dismantled state of the fragrance cartridge is always closed in such a way that no fragrance can exit unused from the cartridge.

Expediently, the housing opening of the housing in the closed position is closed in a fluid-tight manner by means of the actuating element. Alternatively or additionally, in the closed position the element opening is closed in a fluid-tight manner by means of the housing. This advantageously offers a particularly effective possibility to close the element interior in a fluid-tight, in particular fragrance-tight, manner in the closed position.

In a preferred further development of the self-closing fragrance cartridge, a fragrance source for providing a fragrance is present in the element interior. Here, the fragrance in the fragrancing position is able to be directed out from the element interior and sealed in the closed position of the element interior in a fluid-impermeable manner. This means that in the closed position, the element interior is closed in particular in a fragrance-impermeable manner. Thereby, advantageously it is made possible that an uncontrolled contamination of the element interior or respectively of the fragrance source present in the element interior with impurities present in an environment of the fragrance cartridge can be effectively prevented or at least reduced.

In a further advantageous further development of the self-closing fragrance cartridge, the actuating element is mounted displaceably on the housing via a clamping device for generating an actuating force acting on the actuating element. Here, the actuating element is mounted displaceably on the housing via the clamping device in such a way that on displacing of the actuating element from the closed position into the fragrancing position, a restoring force is built up. This advantageously offers a particular possibility, which is easily able to be implemented, to form the self-closing closure mechanism of the fragrance cartridge.

According to a further preferred further development of the fragrance cartridge, an opener is arranged, in a force-transmitting manner, on the actuating element. Expediently, this opener is formed integrally on the actuating element, therefore is formed in the same material and in one piece with the actuating element. The opener, arranged on the actuating element, is actuatable by means of a displacement force for displacing the actuating element from the closed position into the fragrancing position. Expediently, the opener is also actuatable for displacing the actuating element back from the fragrancing position into the closed position. Advantageously, by means of such an opener, the actuating element can be displaced particularly well.

Expediently, the opener is actuatable for displacing the actuating element from the closed position into the fragrancing position on a mounting of the fragrance cartridge on a fragrancer by means of an activating device provided on the fragrancer with the generation of the displacement force. This advantageously makes it possible that a mounting force which must be applied for placing the fragrance cartridge on the fragrancer contains at the same time a portion which corresponds to the activating force. Therefore, advantageously no separate mounting step is necessary, in which the fragrance cartridge is opened separately. This saves time in the mounting of the fragrance cartridge and accordingly also saves time-related mounting costs.

In a further advantageous further development of the self-opening fragrance cartridge, the actuating element is able to be restored automatically into the closed position on a withdrawing of the displacement force by means of the restoring force. Particularly expediently, the actuating element is able to be automatically restored completely on a complete withdrawing of the displacement force by means of the restoring force. The advantage results herefrom that the fragrance cartridge, after a dismantling of the fragrance cartridge from a fragrancer, is automatically closed directly again, so that no fragrance can exit in an undesired manner and unused out from the fragrance cartridge and be lost.

A further preferred further development of the fragrance cartridge makes provision that the actuating element is displaceable relative to the housing in the manner of a slider. Expediently, the actuating element is displaceable in the manner of a slider substantially in a straight line relative to the housing. This advantageously permits a particularly robust design of the actuating element of the fragrance cartridge.

According to a further advantageous further development of the self-closing fragrance cartridge, the actuating element is rotationally displaceable relative to the housing in the manner of a rotary slider. Expediently, the actuating element is rotationally displaceable relative to the housing about a rotation axis in the manner of a rotary slider. Such an actuating element is advantageously displaceable with a particularly small displacement force which, moreover, depends only slightly on a pressure ratio acting on the actuating element.

In a further preferred further development of the self-closing fragrance cartridge, the actuating element is guided on the housing in a perfectly fitting manner. Expediently, the actuating element is guided on the housing in a perfectly fitting manner in the manner of a clearance fit or transition fit. Particularly expediently, a seal is present in the housing interior between the actuating element and the housing. In such a design of the self-closing fragrance cartridge with actuating element and housing coordinated with one another in a perfectly fitting manner, in the closed position of the actuating element it can be ensured particularly effectively that no fragrance can leave the element interior.

A further advantageous further development of the self-closing fragrance cartridge makes provision that the housing has two housing openings and the actuating element has two element openings. Here, in the fragrancing position of the actuating element, each one of the housing openings is arranged in a fluidically communicating manner with in each case a corresponding element opening of the actuating element. Advantageously, a particularly large amount of fragrance can thus be discharged per unit of time from the cartridge in the fragrancing position of the actuating element.

Expediently, the housing has several housing openings and the actuating element has several element openings. Here, each one of the housing openings is arranged in a fluidically communicating manner with in each case a corresponding element opening in the fragrancing position of the actuating element. Advantageously, an even greater amount of fragrance can thereby be released in the fragrancing position of the actuating element.

In an advantageous further development of the self-closing fragrance cartridge, a number of housing openings corresponds to a number of element openings. This offers production-orientated advantages.

According to a further advantageous further development of the self-closing fragrance cartridge, the fragrance source is arranged between the element openings. Expediently, the fragrance source is arranged centrally between the element openings. Thereby, it is advantageously achieved that a fragrance delivered from the fragrance source in the fragrancing position of the actuating element can be distributed particularly uniformly to the element openings.

In addition, the invention relates to a fragrancer for the dispensing of a fragrance. Expediently, the fragrancer serves for the fragrancing of a vehicle interior. The fragrancer has a fragrance inlet on which a fragrance cartridge according to the invention is able to be mounted in accordance with the above description. The fragrance inlet of the fragrancer forms here expediently the outer space of the fragrance cartridge. The fragrancer has an activating device, by means of which the actuating element of the fragrance cartridge, on mounting of the fragrance cartridge, is displaceable from the closed position into the fragrancing position of the actuating element. The advantages, indicated above, of the self-closing fragrance cartridge according to the invention apply in an analogous manner also to the fragrancer according to the invention.

Further important features and advantages of the invention will emerge from the subclaims, from the drawings and from the associated figure description with the aid of the drawings.

It shall be understood that the features mentioned above and to be explained further below are able to be used not only in the respectively indicated combination, but also in other combinations or in isolation, without departing from the scope of the present invention.

Preferred example embodiments of the invention are illustrated in the drawings and are explained in closer detail in the following description, wherein the same reference numbers refer to identical or similar or functionally identical components.

There are shown, respectively schematically

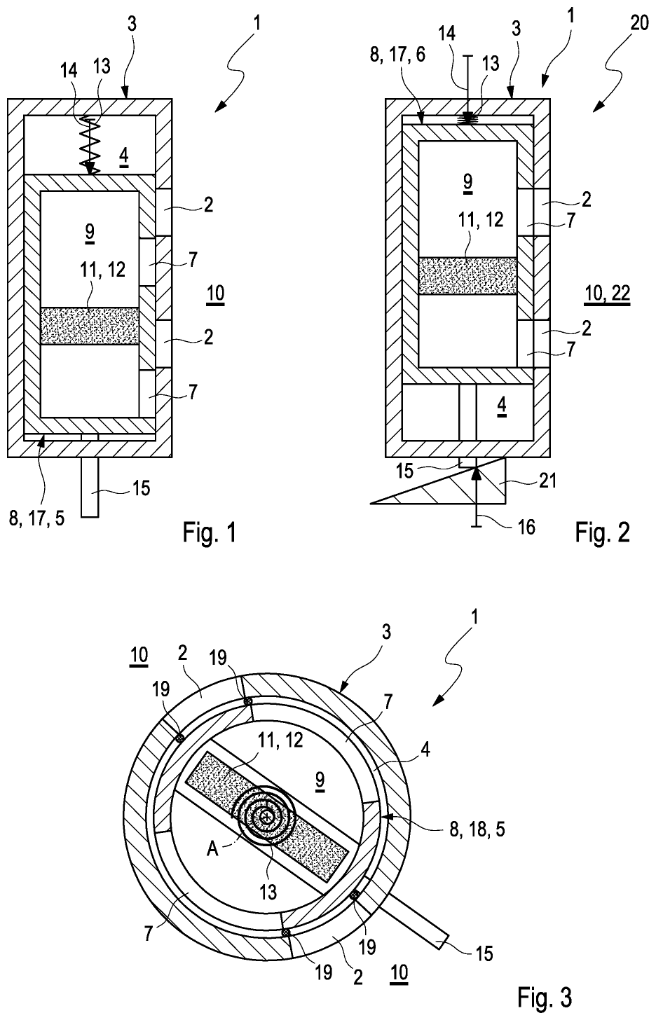

FIG. 1 in a sectional view, an example of a self-closing fragrance cartridge according to the invention, with an actuating element situated in a closed position,

FIG. 2 in a sectional view, an example of a fragrancer according to the invention, on which a fragrance cartridge, shown by way of example, is mounted with an actuating element situated in a fragrancing position,

FIG. 3 a further example of a self-closing fragrance cartridge according to the invention in a sectional view.

In FIG. 1 an example of a self-closing fragrance cartridge 1 according to the invention for a fragrancer 20 is shown in a roughly schematic sectional view. The self-closing fragrance cartridge 1 has a housing 3 on which at least one housing opening 2 is present. In the example shown, two such housing openings 2 are present on the housing 3 of the self-closing fragrance cartridge 1. The housing 3 at least partially delimits a housing interior 4 on the inner side. An actuating element 8 of the self-closing fragrance cartridge 1 is received at least partially in the housing interior 4. The actuating element 8 is displaceable in the housing interior 4 with respect to the housing 3 between a closed position 5 and a fragrancing position 6.

In FIG. 1, the actuating element 8 is situated in the closed position 5. The actuating element 8 at least partially delimits an element interior 9. In the example shown, it can be seen that in the closed position 5 of the actuating element 8, the element interior 9 of the actuating element 8 is closed in a fluid-tight manner by means of the housing 3 of the fragrance cartridge 1. Here, the element interior 9 in the closed position 5 is closed in a fluid-tight manner by means of the housing 3 in such a way that the element interior 9 is expediently closed in a fragrance-tight manner.

In FIG. 2, by comparison, also in a roughly schematic sectional view, a further example of a self-closing fragrance cartridge 1 according to the invention is shown, which is mounted on a fragrancer 2 according to the invention and likewise illustrated by way of example. In the example of FIG. 2, the actuating element 8 of the self-closing fragrance cartridge 1 is situated in the fragrancing position 6. It can be seen that in the fragrancing position 6 the housing opening 2 and the element opening 7 are arranged in a fluidically communicating manner. In the shown example the housing opening 5 and the element opening 7 are arranged in alignment to one another. The fact that the housing opening 2 and the element opening 7 in the fragrancing position 6 are arranged in a fluidically communicating manner is expediently achieved in that the housing opening 2 and the element opening 7 are arranged in an at least partial overlap. In the example shown, the housing opening 2 and the element opening 7 are arranged in complete overlap. Here, in the fragrancing position 6, the housing opening 2 and the element opening 7 are arranged in a fluidically communicating manner in such a way that by means of the housing opening 2 and the element opening 7, the element interior 9 of the actuating element 8 is connected in the fluid-permeable manner with an outer space 10 adjoining the housing 3 on the outer side. This means that in the fragrancing position 6, the housing opening 2 and the element opening 7 can form a fragrance-permeable passage, through which a fragrance 12 can arrive from the element interior 9 into the outer space 10.

With the aid of FIG. 1 it can be seen in addition that in the closed position 5 of the actuating element 8 the housing opening 2 is closed in a fluid-tight manner by means of the actuating element 8. Alternatively or additionally, in accordance with the shown example, the element opening 7 is closed in a fluid-tight manner by means of the housing 3. In the element interior 9 of the actuating element 8 a fragrance source 11 is present for providing a fragrance 12. The fragrance 12 here in the fragrancing position 6 is able to be directed out from the element interior 9, where, on the other hand, the element interior 9 in the closed position 5 is sealed in a fluid-impermeable manner. Such a fluid-impermeable sealing is, in particular, fragrance-tight.

FIG. 3 shows likewise in a roughly schematic manner an example of a self-closing fragrance cartridge 1 according to the invention in a sectional view. According to the example of the self-closing fragrance cartridge 1 shown here, the actuating element 8 is formed in the manner of a rotary slider 18. The actuating element 8, formed as rotary slider 18, is rotationally displaceable relative to the housing 3. In the example shown, the actuating element 8 is rotationally displaceable relative to the housing 3 in the manner of a rotary slider 18 about a rotation axis A. In FIG. 3 in addition it can be seen that a seal 19 is present in the housing interior 4 between the actuating element 8 and the housing 3.

It can be seen in addition from FIGS. 1, 2 and 3 that the actuating element 8 is mounted displaceably on the housing 3 via a clamping device 13 for generating a restoring force 14 acting on the actuating element 8. Such an actuating element 8 can be an elastically deformable spring element. Such a spring element can be a helical spring, a leaf spring, a spiral spring, an elastomer block or similar. The actuating element 8 is mounted displaceably on the housing via the clamping device 13 in such a way that on displacing of the actuating element 8 from the closed position 5 into the fragrancing position 6, the restoring force 14 is built up. Here, in a dismantled state of the fragrance cartridge (shown in FIG. 1 and FIG. 3), the restoring force 14 can hold the actuating element 8 in the closed position 5. It can be seen in addition that an opener 15 is arranged in a force-transmitting manner on the actuating element 8. In the examples shown, the opener 15 is formed integrally for this on the actuating element 8, therefore is designed in the same material and in one piece with the actuating element 8. The opener 15 is activatable by means of a displacement force 16 for displacing the actuating element 8 from the closed position 5 into the fragrancing position 6. The opener can also be activatable for displacing back from the fragrancing position 6 into the closed position 5 of the actuating element 8.

As shown in FIG. 1, the opener 15 is activatable for displacing the actuating element 8 from the closed position into the fragrancing position 6 on mounting of the fragrance cartridge 1 on a fragrancer 20 by means of an activating device 21, provided on the fragrancer 20, with the generation of the displacement force 16. This means that a mounting force applied for mounting the fragrance cartridge 1 on the fragrancer 20 serves on the one hand to position the fragrance cartridge 1 on the fragrancer 22 and, on the other hand, brings about the generating of the displacement force 16. On mounting of the fragrance cartridge 1 on the fragrancer 20, the actuating element 8 is therefore displaced from the closed position 5 into the fragrancing position 6, without further measures being necessary for this, such as a manual displacing of the actuating element 8. Here, with a withdrawing of the displacement force 16 the actuating element 8 is automatically able to be restored into the closed position 5 by means of the restoring force 14. This means that on a dismantling of the fragrance cartridge 1 from the fragrancer 20, the actuating element 8 is automatically restored into the closed position 5 by means of the restoring force 14.

In accordance with FIGS. 1 and 2, the actuating element 8, alternatively to the variant of FIG. 3, is displaceable relative to the housing 3 in the manner of a slider 17. Here, in the example shown, the actuating element 8 is displaceable in the manner of a slider 17 substantially in a straight line relative to the housing 3. Of course, a combination of the examples of FIGS. 1 and 2 and of the example of FIG. 3 is also conceivable, in which the actuating element 8 is displaceable both in a translatory and rotatory manner relative to the housing 3. In addition, alternatively to the examples shown in the figures, the actuating element 8 and the housing 3 can form a cock-like fitting of the fragrance cartridge 1, wherein the actuating element 8 is designed in the manner of a plug of the cock-like fitting.

In accordance with the examples of FIGS. 1 to 3, the actuating element 8 is guided in a perfectly fitting manner on the housing 3. Here, the actuating element 8 is expediently guided on the housing 3 in the manner of a clearance fit or transition fit. The housing 3 and the actuating element 8 can have respectively a rotationally symmetrical shaping. This rotationally symmetrical shaping of the housing 3 and of the actuating element 8 can correspond to that of a hollow cylinder. According to the examples shown, the housing 3 has two housing openings 2 and the actuating element 8 has two element openings 7. Here, each one of the housing openings 2 is arranged in a fluidically communicating manner with in each case a corresponding actuating element opening 7 in the fragrancing position 6 of the actuating element 8. The housing 3 can also have several housing openings 2 and the actuating element 8 can have several element openings 7, which, however, is not shown in the figures for reasons of clarity. Here, also, each one of the housing openings 2 is arranged in a fluidically communicating manner with in each case a corresponding actuating opening 7 in the fragrancing position 6 of the actuating element 8. It can further be seen from the illustrated examples that a number of housing openings 2 corresponds to a number of element openings 7. In addition, it can be seen that the fragrance source 11 is arranged between the element openings 7. For example, the fragrance source 11 is arranged centrally between the element openings 7.

In addition, the invention relates to a fragrancer 20, shown roughly schematically in FIG. 2, which is arranged for dispensing a fragrance 12. The fragrancer 20 can serve for the fragrancing of a vehicle interior. The fragrancer 20 has a fragrance inlet 22, on which a fragrance cartridge 1 according to the invention, shown by way of example in FIGS. 1 to 3 and explained above by way of example, is able to be mounted. It can be seen in FIG. 2 that the fragrance inlet 22 of the fragrancer 20 forms the outer space 10 of the fragrance cartridge 1. The fragrancer 20 comprises in addition an activating device 21, by means of which the actuating element 8 of the fragrance cartridge 1 is displaceable from the closed position 5 into the fragrancing position 6 of the actuating element 8 on mounting of the fragrance cartridge 1. In FIG. 2 the fragrance cartridge 1 is shown in a mounted state, so that accordingly the actuating element 8 is displaced into the fragrancing position 6 by means of the activating device 21.

1. A self-closing fragrance cartridge (1) for a fragrancer (20),
with a housing (3) having at least one housing opening (2), which housing partially delimits a housing interior (4) on the inner side,
with an actuating element (8) arranged at least partially in the housing interior (4) and displaceable with the respect to the housing (3) between a closed position (5) and a fragrancing position (6) and having at least one element opening (7), which actuating element partially delimits an element interior (9),
wherein in the closed position (5) of the actuating element (8), the element interior (9) is closed in a fluid-tight manner by means of the housing (3) of the fragrance cartridge (1),
wherein in the fragrancing position (6), the housing opening (2) and the element opening (7) are arranged in a fluidically communicating manner, in particular in alignment to one another, so that by means of the housing opening (2) and the element opening (7) the element interior (9) is connected in a fluid-permeable manner with an outer space (10) adjoining the housing (3) on the outer side.

2. The self-closing fragrance cartridge (1) according to claim 1,
characterized in that
in the closed position (5) the housing opening (2) is closed in a fluid-tight manner by means of the actuating element (8), and/or the element opening (7) is closed in a fluid-tight manner by means of the housing (3).

3. The self-closing fragrance cartridge (1) according to claim 1 or 2,
characterized in that
in the element interior (9) a fragrance source (11) is present for providing a fragrance (12), wherein in the fragrancing position (6) the fragrance (12) is able to be directed out form the element interior (9) and in the closed position (5) the element interior (9) is sealed in a fluid-impermeable manner.

4. The self-closing fragrance cartridge (1) according to one of claims 1 to 3,
characterized in that
the actuating element (8) is mounted displaceably on the housing (3) via a clamping device (13) for generating a restoring force (14) acting on the actuating element (8), in such a way that on displacing of the actuating element (8) from the closed into the fragrancing position (5, 6), the restoring force (14) is built up.

5. The self-closing fragrance cartridge (1) according to one of the preceding claims,
characterized in that
on the actuating element (8) an opener (15) is arranged in a force-transferring manner, in particular is formed on integrally, which is activatable by means of a displacement force (16) for displacing the actuating element (8) from the closed position (5) into the fragrancing position (6), and in particular back.

6. The self-closing fragrance cartridge (1) according to claim 5,
characterized in that
the opener (15) for displacing the actuating element (8) from the closed—into the fragrancing position (5, 6) on mounting of the fragrance cartridge (1) on a fragrancer (20) is formed in an activatable manner by means of an activating device (21), provided on the fragrancer (20), with the generating of the displacement force (16).

7. The self-opening fragrance cartridge (1) according to claim 5 or 6, with referral back to claim 4,
characterized in that
on a withdrawing of the displacement force (16), in particular to a zero value, the actuating element (8) is able to be automatically restored into closed position (5) by means of the restoring force (14).

8. The self-closing fragrance cartridge (1) according to one of the preceding claims,
characterized in that
the actuating element (8) is displaceable relative to the housing (3) in the manner of a slider (17), in particular in a straight line.

9. The self-closing fragrance cartridge (1) according to one of the preceding claims,
characterized in that
the actuating element (8) is rotationally displaceable relative to the housing (3) in the manner of a rotary slider (18), in particular about a rotation axis (A).

10. The self-closing fragrance cartridge (1) according to one of the preceding claims,
characterized in that
the actuating element (8) is guided on the housing (3) in a perfectly fitting manner, in particular in the manner of a clearance fit or transition fit, wherein preferably in the housing interior (4) a seal (19) is present between the actuating element (8) and the housing (3).

11. The self-closing fragrance cartridge (1) according to one of the preceding claims,
characterized in that
the housing (3) has two housing openings (2) and the actuating element (8) has two element openings (7),
wherein each one of the housing openings (2) is arranged fluidically communicating with in each case a corresponding element opening (7) in the fragrancing position (6) of the actuating element (8).

12. The self-closing fragrance cartridge (1) according to one of the preceding claims,
characterized in that
the housing (3) has several housing openings (2) and the actuating element (8) has several element openings (7),
wherein each one of the housing openings (2) is arranged fluidically communicating with in each case a corresponding element opening (7) in the fragrancing position (6) of the actuating element (8).

13. The self-closing fragrance cartridge (1) according to claim 12,
characterized in that
a number of housing openings (2) corresponds to a number of element openings (7).

14. The self-closing fragrance cartridge (1) according to one of the preceding claims, which referral back to claim 3,
characterized in that
the fragrance source (11) is arranged, in particular centrally, between the element openings (7).

15. A fragrancer (20) for dispensing a fragrance (12), in particular for the fragrancing of a vehicle interior,
with a fragrance inlet (22), on which a fragrance cartridge (1) according to one of the preceding claims is able to be mounted,
with an activating device (21), by means of which the actuating element (8) of the fragrance cartridge (1) on mounting of the fragrance cartridge (1) is displaceable from the closed into the fragrancing position (5, 6) of the actuating element (8).
